(19) United States
(12) Patent Application Publication
(10) Pub. No.: US 2023/0404420 A1
(43) Pub. Date: Dec. 21, 2023

(54) NON-INVASIVE OPTICAL MEASUREMENT OF BLOOD FLOW SPEED

(71) Applicant: The Regents of the University of California, Oakland, CA (US)

(72) Inventors: Yu-Hwa Lo, San Diego, CA (US); Ce Zhang, San Diego, CA (US)

(21) Appl. No.: 18/251,070

(22) PCT Filed: Oct. 26, 2021
(86) PCT No.: PCT/US2021/056715

§ 371 Date: Apr. 28, 2023

Related U.S. Application Data
(60) Provisional application No. 63/107,746, filed on Oct. 30, 2020.

(51) Int. Cl. A61B 5/026; A61B 5/00

(52) U.S. Cl. CPC A61B 5/0261; A61B 5/7246; A61B 5/0059

(57) ABSTRACT

Systems and methods for determining a speed of blood flow in a blood vessel which include interacting light with blood in the blood vessel, measuring, using a light detector, a characteristic related to time variations of electric field of light which interacted with the blood in the blood vessel, and generating a numeric value indicative of the blood flow speed in the blood vessel using an autocorrelation function of the electric field associated with the light which interacted with the blood in the blood vessel.

### RELATED APPLICATIONS

This patent document claims priority to and benefits of U.S. Provisional Patent Application No. 63/107,746 entitled “NON-INVASIVE OPTICAL MEASUREMENT OF BLOOD FLOW SPEED” and filed on Oct. 30, 2020. The entire contents of the before-mentioned patent application are incorporated by reference as part of the disclosure of this patent document.

### TECHNICAL FIELD

The disclosed technology relates to optical systems and, in particular, to methods and devices which enable non-invasive measurements of blood flow speed in blood vessels.

### BACKGROUND

Non-invasive measurement of the arterial blood speed provides important health information such as cardio output and blood supplies to vital organs. The magnitude and change in arterial blood speed are key indicators of the health conditions and development and progression of many diseases. Currently available non-invasive methods of determining arterial blood speed typically require prior knowledge and/or assumptions about geometric or mechanic properties of the arteries. Such information can be difficult to quickly and reliably obtain fora particular person, and the assumptions used can be incorrect for the particular person which makes blood speed measurements using the existing methods time consuming and/or unreliable.

Accordingly, a need exists to provide non-invasive methods of determining arterial blood speed that are free from the mentioned limitations of the currently available technology.

### SUMMARY

The techniques disclosed herein can be implemented in various embodiments to provide systems, methods and devices that, among other features and benefits, can be used to measure flow speed of blood in blood vessels (e.g., arteries or veins) of a body directly and in a non-invasive fashion.

An aspect of the disclosed embodiments relates to a method of non-invasive blood flow speed measurement in a blood vessel that includes illuminating an area of a body comprising the blood vessel using a light source such that light from the light source interacts with blood in the blood vessel. The method further includes receiving light which interacted with the blood in the blood vessel by a light detector. The method also includes generating, by the light detector, one or more signals corresponding to the light which interacted with the blood in the blood vessel received by the light detector, wherein the one or more signals are indicative of temporal variations of an electric field associated with the light received by the light detector. Furthermore, the method includes generating a numeric value indicative of the blood flow speed in the blood vessel using an autocorrelation function of the electric field associated with the light received by the light detector, wherein said generating the numeric value is performed without using any dimension of the blood vessel or any mechanical property of the blood vessel.

Another aspect of the disclosed embodiments relates to a system for non-invasive blood speed measurements that includes one or more light sources configured to produce light to illuminate an area of a body comprising a blood vessel. The system further includes one or more light detectors positioned to receive light subsequent to interaction with blood in the blood vessel and to generate one or more signals corresponding to received light after interaction with the blood in the blood vessel, the one or more signals indicative of temporal variations of an electric field associated with the received light. The system also includes a processor coupled to the one or more light detectors and a memory comprising processor executable code, wherein the processor executable code, upon execution by the processor, causes the processor to: receive information corresponding to the one or more signals generated by the one or more light detectors and determine an estimate of a blood flow speed in the blood vessel using an autocorrelation function of the electric field associated with the received light without using any dimension of the blood vessel or any mechanical property of the blood vessel.

### BRIEF DESCRIPTION OF THE DRAWINGS

FIG. 1 shows a system for measuring blood flow speed according to an example embodiment.

FIG. 2A shows plots of an electric field (E-field) correlation function, determined using a system according to an example embodiment, versus correlation time for different blood flow speeds and a tube inner diameter (ID) of 1.68 mm.

FIG. 2B shows a plot of decorrelation rate versus blood flow speed obtained using the curves shown in FIG. 2A.

FIG. 3A shows plots of an E-field correlation function, determined using a system according to an example embodiment, versus correlation time for different blood flow speeds and a tube inner diameter of 1.14 mm.

FIG. 3B shows a plot of decorrelation rate versus blood flow speed obtained using the curves shown in FIG. 3A.

FIG. 4A shows plots of an E-field correlation function, determined using a system according to an example embodiment, versus correlation time for different blood flow speeds and a tube inner diameter of 0.93 mm.

FIG. 4B shows a plot of decorrelation rate versus blood flow speed obtained using the curves shown in FIG. 4A.

FIG. 5 shows measured dependencies of decorrelation rate versus blood flow speed for different tube IDs.

FIG. 6 illustrates an elementary scattering volume.

FIG. 7 illustrates a cylindrically symmetric 2-dimensional model of a blood vessel used for simplifying the calculations.

FIG. 8 shows examples of calculated and measured characteristic decorrelation rates for different flow speeds and tube inner diameters.

FIG. 9 illustrates a block diagram of a device which can be used to implement, at least in-part, some of the various embodiments disclosed herein.

FIG. 10 shows a flow diagram of an example embodiment of a method of non-invasive blood flow speed measurement in a blood vessel according to the disclosed technology.

### DETAILED DESCRIPTION

The techniques disclosed herein overcome the shortcomings of prior methods and can be implemented in various embodiments to provide non-invasive methods of measuring arterial blood speed without any prior knowledge, information or assumption about the geometric or mechanic properties of the arteries. Methods and system according to the present disclosure allow performing direct measurements of blood flow speed in blood vessels such as, e.g., main arteries of a body based on the diffused light approach described herein.

Oxygenated red blood cells (RBCs) in the blood flow deliver essential nutrients and oxygen to organs and limbs to maintain their homeostatic conditions and proper functions. An oximeter (also referred to as pulse oximeter) can detect the oxygen saturation level of red blood cells by using wavelength-dependent absorption of oxygenated and deoxygenated blood. Such measurements can be done with wearable devices such as, e.g., smart watches. On the other hand, there has been no convenient and non-invasive method to measure the amount of blood supply or blood speed in major arteries.

Reduced blood supply is usually a sign of many diseases and medical conditions. Dehydration, blood clots formation, and other physiological and pathological conditions can cause short-term or long-term changes in the blood speed at a given location of an artery. The information related to the level and/or changes in the blood speed can help early diagnosis and monitoring of diseases characterized by impaired blood flow. Therefore, it is desirable to directly measure the travelling velocity of red blood cells at well-defined locations such as, e.g., the carotid artery to the head, femoral artery to the lower limb, brachial artery to the upper arm, spinal arteries to the spinal cord, renal arteries to the kidneys, hepatic artery to the liver, and pulmonary artery to the lungs, etc. since the arterial blood speed at the well-defined positions provides direct and unambiguous information about blood supply to the sites of health concerns.

Doppler frequency shift of ultrasonic waves offers a non-invasive measurement of the blood flow speed at the well-defined position. However, acoustic measurements require close physical contacts of the ultrasound transducers with the tissue to allow efficient coupling of the acoustic wave, and gel is needed to assure the quality and reliability of the contact. Furthermore, the measured signal is highly dependent on the angle between the probe and the blood flow direction. The most convenient and ergonomic direction is to have the transducer perpendicular to the blood vessel. However, this arrangement will produce no Doppler shift signal, causing to use different arrangement(s) and thus making the measurements less convenient in a homecare or nursing home setting for self-administered operation. In this regard, an optical method is potentially more desirable because a light wave can enter the tissue via free space coupling and, once in the tissue, the light wave travels diffusively. In addition, the optical hardware including semiconductor light sources and detectors is more compact than acoustic transmitters and receivers.

Dynamic scattering of laser light by red blood cells can be used to measure blood speed in blood vessels near the tissue surface. Similar to the Doppler effect of sound propagation, the frequency of the light scattered by moving particles is also shifted. Although the operation principle seems to be straightforward, the physical model of the “optical Doppler device” is based on the key assumption that the light is scattered only once by a moving object (e.g., a traveling red blood cell) and the rest of the scatterings are produced by quasi-static objects such as, e.g., skin or fat. This assumption may be reasonable for probing blood flow in near-surface tissues, but is not valid for major arteries where around 40% of the volume of the blood vessel with a diameter of a few millimeters is occupied by traveling red blood cells. Under the approximation of a single light scattering by moving particles, the detected scattered light will display a frequency shift proportional to the velocity of the particles and the magnitude of the frequency shift can be detected using, e.g., the homodyne coherent detection technique. However, the homodyne coherent detection technique cannot be applied to multiple scatterings by a large number of moving particles. In such situations, the frequency spectrum of the light wave is broadened, making the interpretation of signal difficult. Because of this limitation, the current techniques based on optical Doppler shift measurements have been limited to measuring the blood flow in microflow channels near the skin although the blood supply by major arteries produces more valuable information for health and disease conditions. In addition, the laser Doppler flowmetry measures only the relative flow speed instead of the absolute flow speed.

To measure the blood flow speed in major arteries, methods and systems according to the present disclosure extend the technique of optical scattering method by taking the effect of multiple scatterings by red blood cells into account. Instead of measuring the Doppler shift in the optical frequency, methods and systems according to the disclosed technology use measurements of the decorrelation time of the reflected or transmitted light to obtain the arterial blood flow speed. An unexpected result provided by the methods according to the present disclosure is that for main arteries, the decorrelation time is inversely proportional to the RBC speed, does not depend on a size (e.g., a diameter) of the artery, and we can measure its value without any prior knowledge about the anatomy and/or mechanical properties of the artery tissues. An optical setup according to an example embodiment includes a single semiconductor laser and a single detector, both coupled to a multi-mode fiber bundle.

Upon entering a biologic tissue, light has a very short scattering mean free path and quickly becomes diffusive. Modelling of light propagation in this regime has led to the development of oximeters back in 70s and many researchers have utilized the diffused light to construct images through highly diffusive biological media, such as reconstructed breast cancer images. The standard configuration for diffused light spectroscopy includes a point source and a point detector, and the measured correlation function depends on the relative position between the source and the detector. The method of diffused light correlation spectroscopy has been used in blood perfusion measurement, including brain circulations. The setup uses a single-mode fiber-coupled detector to allow single-mode transmission of two orthogonal light polarizations. The single spatial mode yields the best signal-to-noise ratio since it detects one speckle. However, this setup produces very low light intensity and requires a single photon detector (e.g., a single-photon avalanche detector (SPAD) or a photomultiplier tube (PMT)), making the setup quite sophisticated and subject to interference and stray light.

Systems according to some example embodiments can use multimode fiber and a regular photodetector to detect temporal fluctuations of the light intensity. Such systems provide a simple and robust setup that is relatively insensitive to the optical alignment between the fiber bundle and the tissue and allow reliably measuring absolute values of speed of red blood cells traveling in a blood vessel which, e.g., has a diameter on the order of millimeters and is embedded in a layer of tissue.

An example implementation of the disclosed technology is demonstrated below using a phantom that includes an intralipid hydrogel to model a biological tissue and a hollow glass tube embedded within the phantom with human blood flowing within the tube to model a blood vessel (e.g., an artery). The correlation function of the measured photocurrent was used to find the electric field correlation function via the Siegert Relation. We have shown that the characteristic decorrelation rate (i.e., the inverse of the decoherent time) is linearly proportional to the blood speed and independent of the diameter of the tube. This striking property can be explained by an approximate analytic solution for the diffused light equation in the regime where the convective flow is the dominating factor for decorrelation.

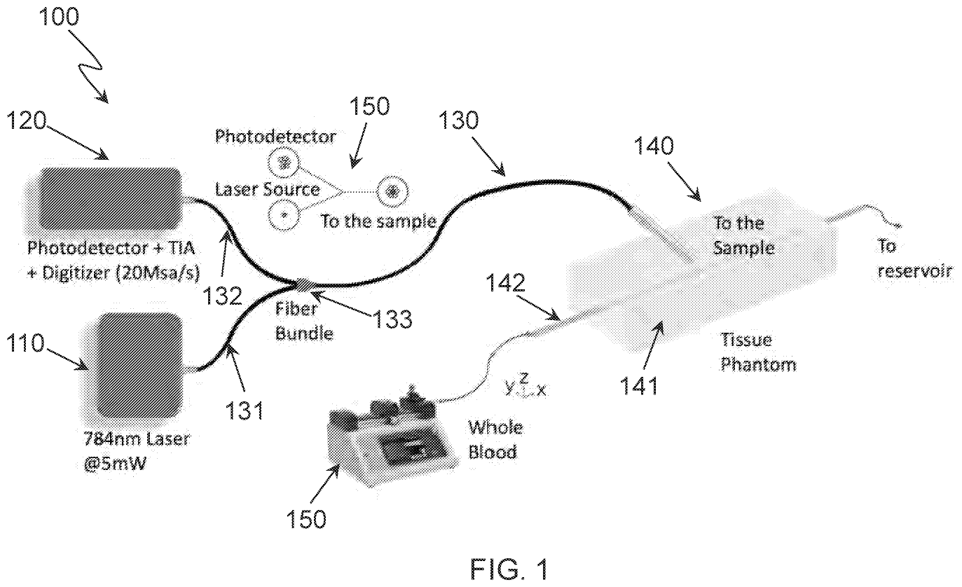

FIG. 1 shows a system 100 for measuring blood flow speed according to an example embodiment. The system 100 includes a light source 110, a photodetector 120, and a multi-core reflection fiber bundle probe 130. In some example embodiments, the light source is a laser light source. According to some example embodiments, the laser light source is a fiber-coupled laser diode configured to emit light on wavelengths at or about 784 nm or 785 nm which may be biased slightly above its threshold current to produce an output of, e.g., −5 mW. In some example embodiments, the multi-core reflection fiber bundle probe 130 may comprise a center core surrounded by several (e.g., 6) peripheral cores to couple the input and reflected light. The center core (referred to as an illumination core or illumination fiber) may be used to deliver light produced by the light source 110 to the target blood vessel(s) and the peripheral cores (referred to as detection cores or detection fibers or detection fiber bundle) may receive light coming from the target blood vessel(s) and transmit that light towards the photodetector 120. Outputs of the peripheral cores may be merged into a single output and coupled to the photodetector 120. In FIG. 1, the arm 131 of the fiber bundle probe 130, which includes the illumination fiber, is coupled to the light source 110 and the arm 132 of the fiber bundle probe 130, which includes fibers of the detection fiber bundle, is coupled to the photodetector 120. The arms 131 and 132 of the fiber bundle probe 130 are merged at the junction 133. Schematic diagram 150 in FIG. 1 illustrates an example arrangement of the fibers within the fiber bundle 130.

In some example embodiments, the photodetector 120 may include a transimpedance amplifier and a digitizer (e.g., a digitizer capable of acquiring data samples at a 20 million samples per second (Msa/s) rate). According to some example embodiments, the photodetector 120 is a silicon avalanche photodetector (APD) with an integrated transimpedance amplifier (e.g., Thorlabs APD410A) which can achieve a transimpedance gain of 500 kV/A at 10 MHz bandwidth. The APD multiplication gain (M) may be set to be the lowest available (e.g., M˜10) in some example embodiments. The output signal from the APD photoreceiver may be sampled by a data acquisition board (e.g., Advantech PCI-E 1840) at a sampling rate of, e.g., 20 Msa/s, thus yielding a Nyquist bandwidth of 10 MHz.

In some example embodiments, the total data acquisition time during a blood velocity measurement may be on the order of seconds (e.g., 25 seconds). The total data acquisition duration may be split into shorter time intervals which a referred to as recording sections. For example, each 5 ms duration may be treated as one recording section, so 25 seconds of measurement will produce 5,000 sections for analysis and noise cancellation. In the data presented next, data averaged over 25 seconds was used for the mean and data averaged over 1 second was used to determine the variations shown in error bars. For a blood flow which is pulsive, the pulse period is on the order of seconds, but the measurement interval (interval between the data samples) used by some example embodiments is on the order of microseconds thus allowing to measure the instantaneous flow speed of blood corresponding to the systolic and diastolic cycles.

To illustrate operating principles and methods of the system 100, blood flow speed measurements were performed using a tissue phantom 140. During those measurements, the laser light generated by the light source 110 of the system 100 was transmitted by the illumination fiber of the reflection fiber bundle probe 130 to the tissue phantom 140. The laser light illuminated the tissue phantom surface at about 45-degrees angle to prevent specular reflection from the surface and the scattered diffused light was collected through the detection fibers of the reflection fiber bundle probe 130 which were coupled to a photoreceiver of the photodetector 120. A glass tube that emulated a blood vessel was embedded in the tissue phantom 5 mm below the surface of the phantom. The reflection fiber bundle probe 130 was coarsely aligned to the position of the glass tube in the tissue phantom.

For the tissue phantom 140, we used intralipid as the scattering agent to mimic the tissue by making its optical scattering coefficient μₛ and anisotropy factor g similar to the values for a real tissue. Because optical properties of intralipid are similar to those of the bilipid membrane of cells, intralipid is commonly used to simulate tissue scattering. In addition, the absorption coefficient of intralipid is low and its refractive index is close to that of soft tissue.

The tissue phantom was created using intralipid (Sigma-Aldrich 20%) in gelatin gel. One percent concentration of intralipid was chosen to achieve a scattering coefficient of around 10 cm⁻¹ at 784 nm. The phantom was initially prepared at 90° C., and then poured into a mold where a glass tube was pre-inserted at a depth of 5 mm from the phantom surface (measured from the center of the tube). After synthesis, the liquid phantom was immediately placed into a freezer at −18° C. for 30 minutes for rapid solidification to prevent sedimentation of intralipid to achieve a uniform scattering property. Then the sample was placed in a refrigerator at 6° C. for 30 minutes to further solidify the sample. The subsequent blood flow velocity measurement experiment was usually completed within 30 minutes after the phantom fabrication to prevent evaporation-induced dehydration of the phantom, which could reduce the surface height of the phantom. Glass tubes of three different inner diameters were used to mimic the arteries. Tygon tubing was connected to both ends of the glass tube, with one end connected to a 10 mL syringe mounted on a syringe pump (150 in FIG. 1) and the other end connected to a reservoir.

12 mL of human whole blood collected on the same day of the experiment was purchased from the San Diego Blood Bank. Ethylenediamine tetraacetic acid (EDTA) was added to the blood sample to prevent coagulation.

Blood flow speed measurements were performed for three tube inner diameters: 0.93 mm, 1.14 mm, and 1.68 mm. The tube diameters were measured by a digital microscope to assure high accuracy. For each tube diameter, different flow rates of the blood through the tube (which correspond to different speeds of the blood flow through the tube) were tested.

We have used the setup shown in FIG. 1 to measure the photocurrent correlation defined as

$$\frac{〈 i ( t ) i ( t + τ ) 〉}{{〈 i ( t ) 〉}^{2}}$$

where i(t) is the photocurrent at time “t” and [symbol] [symbol] is the ensemble average. For a 25 second measurement that can be divided into 5,000 5 ms long sections, the ensemble average is the average of these 5,000 sections. τ is the time delay between the instantaneous photocurrents, and is the variable for the correlation function. Because the photocurrent dependence vs. time is correlated with itself, the correlation function of the photocurrent is an autocorrelation function.

One important relation is to convert the photocurrent correlation into the normalized electric field correlation function g₁ represented by equation (1):

$${g}_{1} ( τ ) = \frac{〈 E ( t ) {E}^{*} ( t + τ ) 〉}{〈 E ( t ) {E}^{*} ( t ) 〉} \qquad ( 1 )$$

Similarly to the photocurrent case, because the dependence of electric field vs. time is correlated with itself in Eq. (1), the correlation function of the electric field is an autocorrelation function.

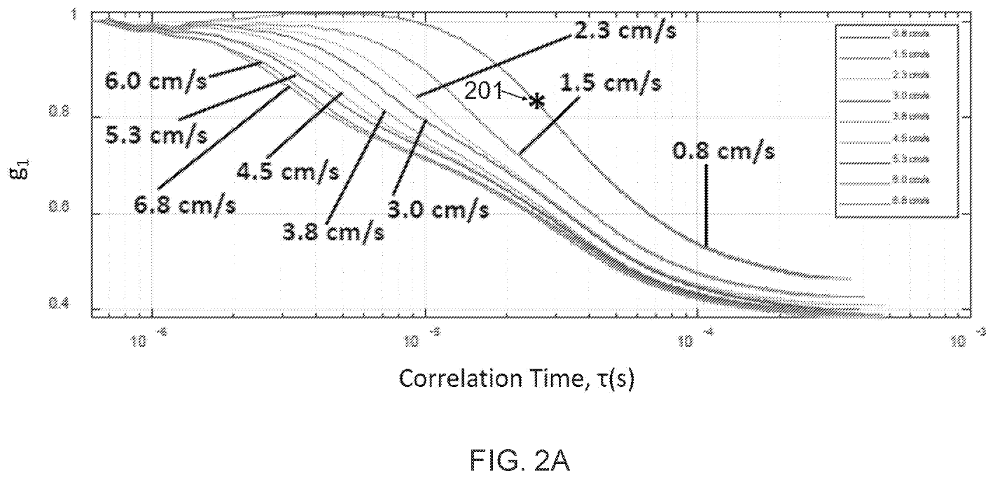

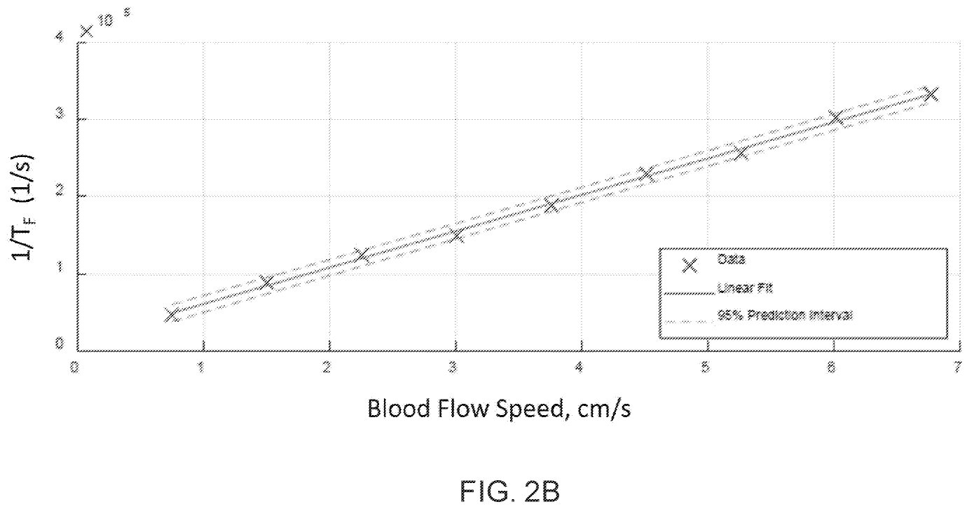

Using the Siegert Relation, we can obtain a relation between the magnitude of g₁(τ) and the photocurrent correlation as shown in equation (2):

$$\frac{〈 i ( t ) i ( t + τ ) 〉}{{〈 i ( t ) 〉}^{2}} = 1 + \frac{1}{N} {\vert {g}_{1} ( τ ) \vert}^{2} + \frac{e δ ( τ )}{〈 i ( t ) 〉} \qquad ( 2 )$$

where i(t) is the measured photocurrent, N is the number of spatial modes coupled into the detection fiber bundle and collected by the detector, δ(τ) is a delta function, and e is the electron charge. The last term in equation (2) represents the shot noise.
g₁ under different flow speeds of blood is shown in semi-log plots in FIGS. 2A, 3A, and 4A, where each figure shows measurements performed with a different tube diameter. The x-axis of each plot in FIGS. 2A, 3A, and 4A is the logarithmic of τ
   g₁ vs. ln(τ) curve shows a characteristic analogous to the “Fermi-Dirac distribution function” if we treat ln(τ) as “energy”; (b) at high blood flow speed, the curve behaves like a superposition of two Fermi-Dirac distribution functions, one at lower “energy” and another at higher “energy.” These characteristics become more apparent if we take the derivative of g₁ with respect to ln(τ), showing that the “Fermi level,” ln(T_{F}), occurs at the inflection point where the magnitude of slope reaches the maximum.

By examining the features of the measured electric field correlation function g₁, we can extract the “Fermi energy, ∈_{F}” or ln(T_{F}). In the high blood speed regime where the |g₁| plot shows two superimposed Fermi-like functions, we chose the inflection point of the lower energy function (i.e., in the regime of smaller ln(τ) values). We will elucidate the reasons for such a choice below. Essentially, each of the two superimposed Fermi-like functions represents a corresponding regime of light scattering mechanisms.

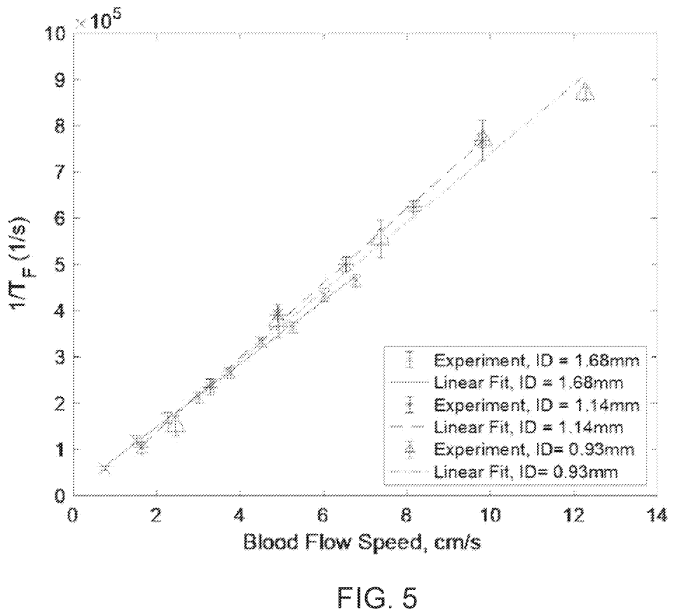

FIGS. 2B, 3B, and 4B show the plot of 1/T_{F} (which is equivalent to e^{∈}^{_{F}}) versus the blood flow velocity for different tube diameters. Amazingly, we obtain a simple linear relation between 1/T_{F} and the blood speed. More interestingly, we have found that the 1/T_{F} versus speed curve is independent of the tube diameter. As shown in FIG. 5, the three curves of 1/T_{F} versus blood flow speed measured using different tube diameters completely overlap and can be represented by one simple relation independent of the tube diameter. This result demonstrates that systems and methods according to the disclosed technology can be used to measure the blood flow speed in blood vessels (e.g., arteries or veins) of different sizes without having to know the exact dimensions of the blood vessel (e.g., its diameter). This discovery is very important in practical applications because it shows that from the g₁(τ) curve, which can be obtained from the correlation of the photocurrent, we can obtain the speed of the blood flow directly for different blood vessels at a given position without any prior knowledge of the anatomy of the blood vessel. The discovery of this important relation requires a sound physical foundation, to rule out the possibility for being simply coincidental. The physical model and mathematical analysis will be discussed below.

FIG. 2A shows plots of an E-field correlation function (g₁), determined using a system according to an example embodiment, versus correlation time r(s) for different blood flow speeds and a tube inner diameter (ID) of 1.68 mm. FIG. 2B shows a plot of decorrelation rate (1/T_{F}) versus blood flow speed obtained using the curves shown in FIG. 2A. By taking the inverse of the correlation time at the first inflection point for each curve shown in FIG. 2A, the decorrelation rate (1/T_{F}) is plotted versus the flow speed in FIG. 2B. The first inflection point of the curve corresponding to the blood speed value of 0.8 cm/s in FIG. 2A is shown by the asterisk 201 on the curve. The scattered data set with error bars in FIG. 2B represents the 95% confidence interval over 25 measurements, each for a duration of 1 second. The solid line in FIG. 2B is a linear fit of the measured data.

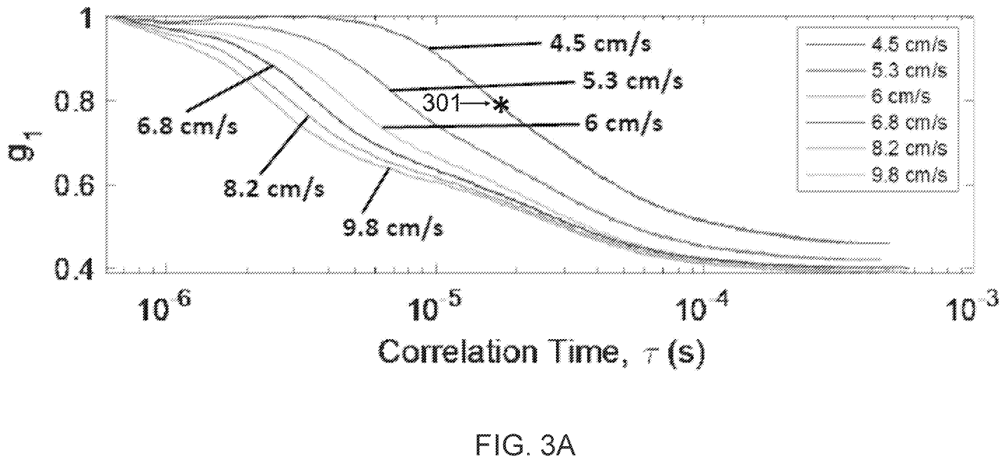

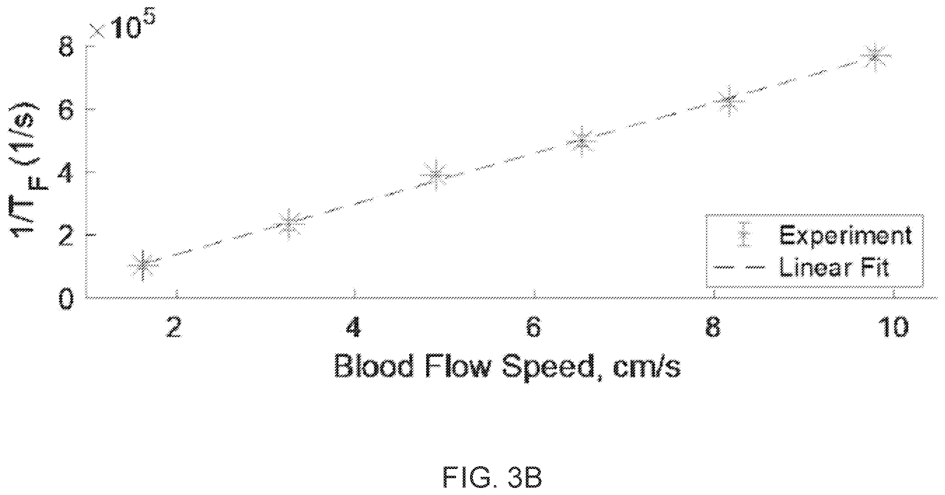

FIG. 3A shows plots of an E-field correlation function (g₁), determined using a system according to an example embodiment, versus correlation time τ(s) for different blood flow speeds and a tube inner diameter of 1.14 mm. FIG. 3B shows a plot of decorrelation rate (1/T_{F}) versus blood flow speed obtained using the curves shown in FIG. 3A. By taking the inverse of the correlation time at the first inflection point for each curve shown in FIG. 3A, the decorrelation rate (1/T_{F}) is plotted versus the flow speed in FIG. 3B. The first inflection point of the curve corresponding to the blood speed value of 4.5 cm/s in FIG. 3A is shown by the asterisk 301 on the curve. The scattered data set with error bars in FIG. 3B represents the 95% confidence interval over 25 measurements, each for a duration of 1 second. The dashed line in FIG. 3B is a linear fit of the measured data.

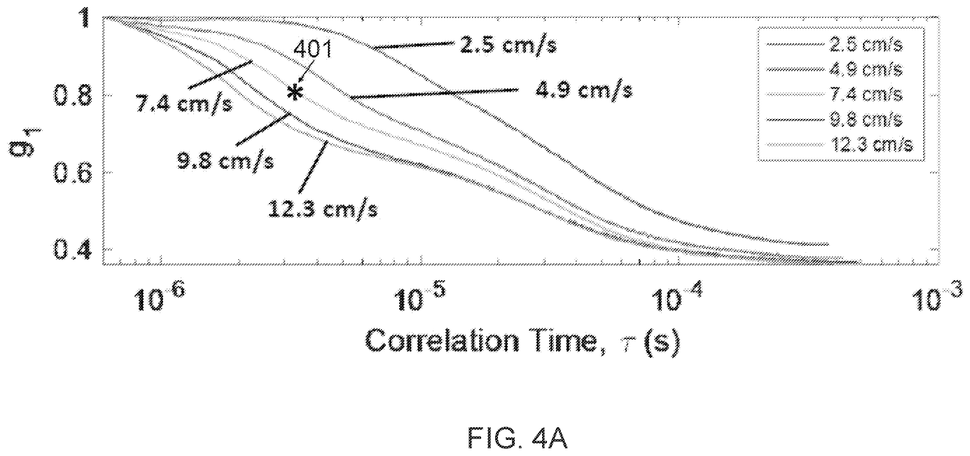

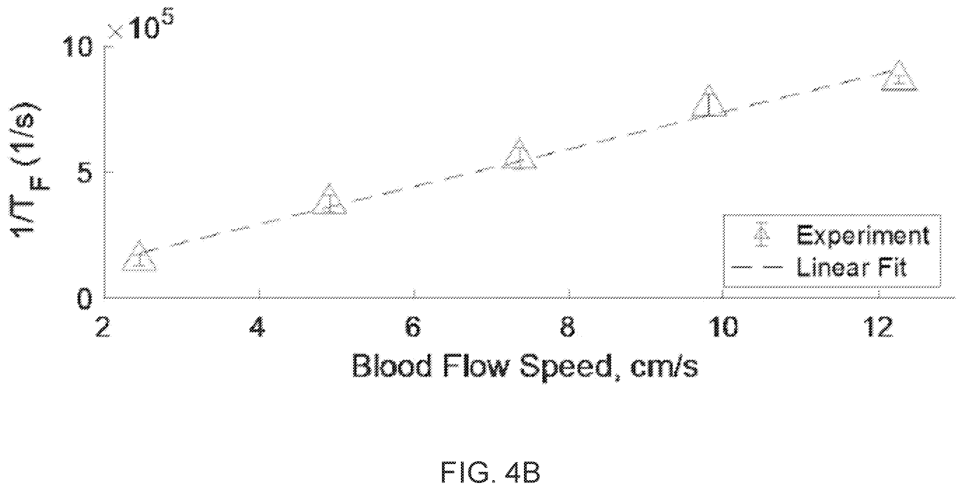

FIG. 4A shows plots of an E-field correlation function (g₁), determined using a system according to an example embodiment, versus correlation time r(s) for different blood flow speeds and a tube inner diameter of 0.93 mm. FIG. 4B shows a plot of decorrelation rate (1/T_{F}) versus blood flow speed obtained using the curves shown in FIG. 4A. By taking the inverse of the correlation time at the first inflection point for each curve shown in FIG. 4A, the decorrelation rate (1/T_{F}) is plotted versus the flow speed in FIG. 4B. The first inflection point of the curve corresponding to the blood speed value of 7.4 cm/s in FIG. 4A is shown by the asterisk 401 on the curve. The scattered data set with error bars in FIG. 4B represents the 95% confidence interval over 25 measurements, each for a duration of 1 second. The dashed line in FIG. 4B is a linear fit of the measured data.

FIG. 5 shows combined plots of dependencies of the decorrelation rate (1/T_{F}) versus blood flow speed for different tube IDs (1.68 mm, 1.14 mm, and 0.93 mm). The plot in FIG. 5 combines the plots shown in FIGS. 2B, 3B, and 4B. The scattered data set with the error bar represents the 95% confidence interval over 25 measurements, each for a duration of 1 second. The line plots are linear fits of the measured data. The fact that dependencies for different tube IDs collapse into one single line demonstrates the existence of a simple relation between the decorrelation rate and the blood speed and demonstrates that the relation is independent of the tube diameter.

In order to understand the underlining physics of the relation between the inverse of characteristic decorrelation time, 1/T_{F}, and the flow speed of the blood, we describe the physical model and mathematical formulation for our experiment below. We will outline the key steps and, through approximations, produce an analytical relation between the blood speed and the decorrelation time.

Two approaches are typically taken to model light propagation in a strongly light-scattering biological medium. In one approach, we start with the wave equation and introduce the scattering and absorption characteristics along the optical path. Despite its rigor in the mathematical formulation, to make the result useful, approximations have to be made to make the problem solvable. Twersky's theory and Dyson's equation fall into this category. In another approach, we can formulate the problem in a transport equation that deals with photon energy transport. These two methods eventually give rise to the same result. Herein, we take the second approach because of its relative simplicity.

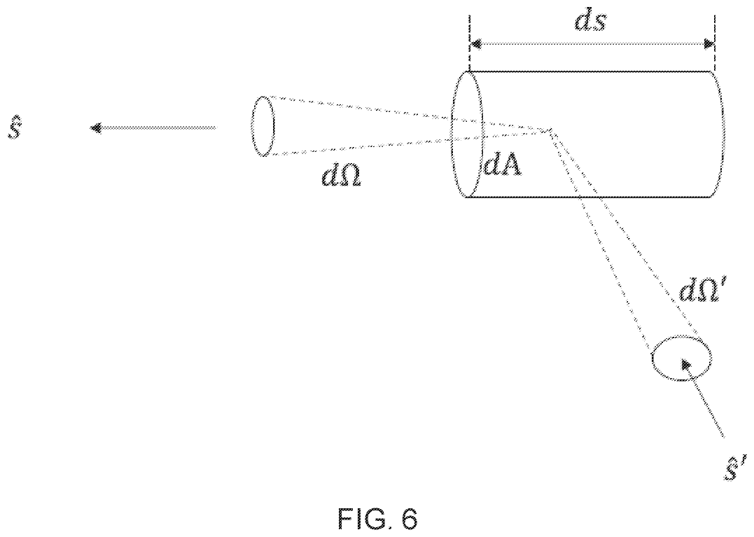

FIG. 6 illustrates an elementary scattering volume for Eq. (3).

The governing equation is a radiative transfer equation (RTE) in the following form:

$$\frac{dI ( \vec{r} , \hat{s} )}{d s} = - ρ {σ}_{t} I ( \vec{r} , \hat{s} ) + ρ {σ}_{s} {\int }_{4 π} p ( \hat{s} , {\hat{s}}^{′} ) I ( \vec{r} , {\hat{s}}^{′} ) d {Ω}^{′} + S ( \vec{r} , \hat{s} ) \qquad ( 3 )$$

in which I is the light-specific intensity with a unit Wm⁻²sr⁻¹, ρ is the particle concentration, σₛ is the scattering cross section, σₜ is the total scattering cross section which is a sum of the scattering cross section and an absorption cross section, p(ŝ,ŝ′) is the normalized differential scattering cross section which is sometimes called the phase function and it is unitless, and S( [symbol] ,ŝ) is the source intensity which has a unit of Wm⁻³sr⁻¹.

FIG. 6 illustrates the orientations and coordinates for an infinitesimal scattering volume. Equation (3) contains 5 coordinates: x,y,z,θ,ϕ where x, y, z define the position vector [symbol] of the light intensity, and θ,ϕ represent the beam's propagation direction. The unit vectors ŝ′ and ŝ represent the direction of the incident light and the propagation direction after scattering, respectively.

Solving equation (3) would produce the full solution for the light scattering problem for any scatter concentration. However, equation (3) is very complicated to solve. Fortunately, in most biological samples, the scatter density is so high that photons quickly lose the memory of their path histories after multiple scatterings and it can be justified to assume the light intensity depends on its position (x,y,z) with a slight flux flow in the direction of propagation (θ,ϕ). This would lead to a simplification of the problem, and the average light intensity could be described by the diffusion equation which is only dependent on the position (x,y,z). Mathematically, it means that we can expand the light intensity in spherical harmonics by keeping the zero order and first order term.

$$I ( \vec{r} , \hat{s} ) \approx \frac{1}{4 π} U ( \vec{r} ) + \frac{3}{4 π} \vec{F} ( \vec{r} ) \cdot \hat{s} \qquad ( 4 )$$

where the first term is the average intensity, and the second term is the small photon flux in the direction of propagation. Applying the diffusion approximation to the RTE, we can obtain a steady state diffusion equation (5) to model light propagation in a diffusive medium when the scatters are not moving. As light needs to be scattered multiple times to become diffusive, an important requirement for the diffusion approximation is that the cross section for light scattering is much stronger than the cross section for light absorption. A problem would arise when dealing with light intensity close to the boundary since the light is highly directional at the boundary, violating the diffusive condition. Different boundary conditions have been explored to resolve this problem, including adoption of an extended boundary condition which uses Taylor expansion to convert the Robin boundary condition into a Dirichlet type boundary condition to simplify the solution for Eq. (5)

[D∇²−μₐ]U( [symbol] )=−S( [symbol] ) (5)

In Eq. (5), D=⅓μₛ′ is the photon diffusivity with a unit of meter and μₛ′ is the reduced scattering coefficient, μₐ is the absorption coefficient with a unit m⁻¹, S( [symbol] ) is the source (unit of Wm⁻³) that depends only on the location but not on the propagation direction, different from S( [symbol] ,ŝ) in equation (3).

When the scatters exhibit motions, the scattered intensity would include time as a parameter. For statistical optics, it is natural to use field correlation function to capture this dynamic process. Here, we are still talking about the steady state response of the system so that the time dependence can be represented as a parameter in the differential equation.

We start with the case of a single scattering event by a moving object. The normalized single scattering function can be written as:

$${g}_{1}^{s} ( τ ) = \frac{〈 E ( t ) {E}^{*} ( t + τ ) 〉}{〈 E ( t ) {E}^{*} ( t ) 〉} = exp ( - \frac{1}{6} {q}^{2} 〈 Δ {r}_{l}^{2} ( τ ) 〉 ) \qquad ( 6 )$$

where g₁^{s} is the single scattering normalized correlation function, E is the scalar electric field of light, q is the photon momentum transfer by each scattering, [symbol] Δrᵢ²(τ) [symbol] is the root mean square (RMS) displacement of the scatters in a duration of τ. The above equation describes the electric field correlation function due to single scattering. This single scattering function can be incorporated into the radiative transfer equation, yielding the so-called correlation transfer equation which is the dynamic counter part of the static radiative transfer equation:

$$\frac{d {G}_{1} ( \vec{r} , \hat{s} , τ )}{d s} = - ρ {σ}_{t} {G}_{1} ( \vec{r} , \hat{s} , τ ) + ρ {σ}_{s} {\int }_{4 π} p ( \hat{s} , {\hat{s}}^{′} ) {g}_{1}^{s} ( \hat{s} , {\hat{s}}^{′} , τ ) I ( \overline{r} , {\hat{s}}^{′} ) d {Ω}^{′} + S ( \vec{r} , \hat{s} ) \qquad ( 7 )$$

Similarly, the diffusion approximation can also be applied to obtain the correlation diffusion equation which is again the counterpart of the static diffusion equation described previously. Equation (8) is the correlation diffusion equation. The diffusion equation can then be solved with appropriate boundary conditions and compared with experimental results.

$$[ D {∇}^{2} - {μ}_{a} - \frac{1}{3} {μ}_{s}^{′} {nk}_{0}^{2} 〈 Δ {r}^{2} ( τ ) 〉 ] U ( \vec{r} ) = - S ( \vec{r} ) \qquad ( 8 )$$

In Eq. (8), n is the refractive index of scattering media, k₀ is the wavevector in vacuum, μₛ′ is the reduced scattering coefficient which is the scattering coefficient modified due to scattering anisotropy. For isotropic scattering, the scattering coefficient would be the same reduced scattering coefficient.

For the purpose of theoretical calculations, we assume a plane wave illumination at a surface illuminated by a light source of a system according to the disclosed technology. Despite this simplification, such approximation can yield satisfactory results in good agreement with the experiment. In some example embodiments, a single detector (e.g., the photodetector 120 in FIG. 1) covers an area defined by the numerical aperture of, e.g., 6 hexagonally arranged multimode fibers of the detection fiber bundle surrounding the illumination fiber (see, e.g., the diagram 150 in FIG. 1). Assuming the blood vessel is cylindrically symmetric, we used a 2D model.

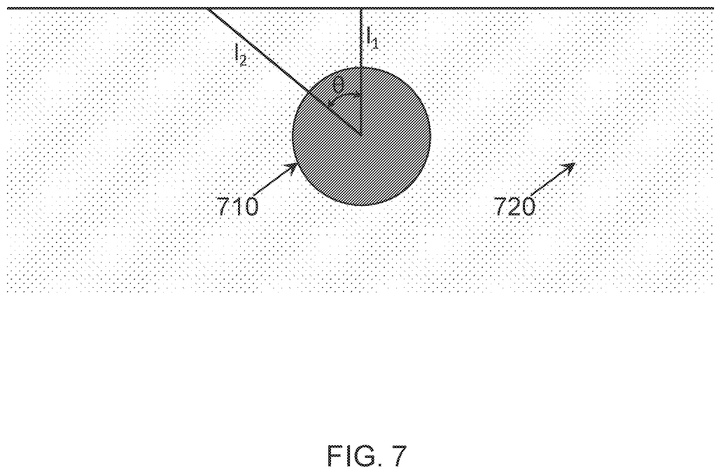

FIG. 7 illustrates a cylindrically symmetric 2-dimensional model of a blood vessel used for simplifying the calculations. The circle 710 represents a cross section of a blood vessel with parameters: Dᵢₙ, W₁, k₁, μₐⁱⁿ, μ′ₛⁱⁿ. The area 720 outside the circle represents a static scattering media, i.e., tissue with parameters: Dₒᵤₜ, W₀, k₀, μₐ^{out}, μ′ₛ^{out}. We adopt cylindrical coordinates and define the center of the vessel as the origin.

We can reduce Eq. (8) into the following diffusion equation

$$[ {∇}^{2} + {k}^{2} ] {G}_{1} ( \vec{r} , τ ) = - \frac{S ( \vec{r} )}{D} , \qquad ( 9 )$$

where

k=jW(τ). (10)

Since the scattering parameters are different inside and outside the blood vessel, two sets of parameters are used. The parameters inside the blood vessel are represented using subscript 1, i.e., k₁=jW₁(τ). The parameters outside the blood vessel are represented using subscript 0, i.e., k=jW₀(τ).

$${W}_{1} = \sqrt{\frac{1}{{D}_{in}} [ {μ}_{a}^{in} + \frac{1}{3} {μ}_{s}^{′} {k}_{λ}^{2} 〈 Δ {r}^{2} ( τ ) 〉 ]} \qquad ( 11 )$$

$${W}_{0} = \sqrt{\frac{{μ}_{a}^{o u t}}{{D}_{o u t}}} \qquad ( 12 )$$

To solve Eq. (9), we need to first find the mean squared displacement [symbol] Δr²(τ) [symbol] . The mean square displacement for RBCs includes Brownian, shear induced diffusion and convective motion. We can represent the position variation of RBCs over a given time interval as:

[symbol]

[symbol]

Δr²(τ) [symbol] =6D_{α}τ+V̅²τ² (13)

where the first term in equation (13) is caused by Brownian and shear induced diffusions and the second term by convection. For RBCs, the diffusion by Brownian motions is much smaller than the shear induced diffusion. The flow of RBCs inside arteries can be modeled by a laminar flow and the diffusion coefficient can be represented as

$${D}_{α} = {α}_{s} \vert \frac{\partial {v}_{R B C}}{\partial r} \vert = \frac{4}{3} {α}_{s} \frac{{V}_{max}}{a} \qquad ( 14 )$$

The radial dependent shear rate is usually replaced by its average value due to the fact that multiple scattering will lead to an ensemble averaging across all radial locations. αₛ is a parameter describing the interaction strength among blood cells due to shear and its value has been measured experimentally. For tissue blood perfusion, the slow flow speed and small blood vessel diameter make the diffusive motion the dominant effect compared to the convective motion. The situation is reversed, however, for main arteries where the vessel inner diameter is of, e.g., millimeter size and the blood flow speed is, e.g., several centimeters per second. In such cases, the convective motion becomes the dominant effect. Hence in our analysis, we have ignored the diffusion contribution and included only the convective flow contribution in Eq. (13).

We can then write the general solution of the correlation function in equation (9) as follows,

G₁( [symbol] ,τ)=G₁ⁱⁿ( [symbol] ,τ)+G₁^{sc}( [symbol] ,τ) (15)

The first term in equation (15) is the inhomogeneous solution and the second term is the homogeneous solution for equation (9) in the absence of the source. The diffusion equation under a given boundary condition can be solved in polar coordinates and the method of separation of variables. For an approximate analytic solution, we kept only the zeroth order term since all higher order terms are insignificant compared with the zeroth order term. A continuity boundary condition is applied at the cylinder interface between the blood vessel and the surrounding tissue. The air-tissue surface is ignored for simplicity in the solution. After solving the differential equation with some approximations based on the numerical value of actual tissue and blood cell scattering properties, the normalized correlation function g₁ can be written in the form of equation (16), inspired from the Fermi-Dirac function in semiconductor physics (i.e., by performing the transformations: ln(τ)=∈, ln(T_{F})=E_{F}, equation (16) is similar to the Fermi-Dirac function).

$${g}_{1} ( r , a , θ , τ ) = \frac{1 - {g}_{1} ( r , a , θ , ∞ )}{1 + τ / {T}_{F}} + {g}_{1} ( r , a , θ , ∞ ) \qquad ( 16 )$$

$$\frac{1}{{T}_{F}} \approx V n {k}_{0} \sqrt{\frac{{μ}_{s}^{′ in}}{3 {μ}_{a}^{in}}} \qquad ( 17 )$$

where V n k₀ is the wavevector of 784 nm light in vacuum, μ′ₛⁱⁿ
m⁻¹), and μₐⁱⁿ is the absorption coefficient of blood (m⁻¹). If we define T_{F}

1/T_{F}

speed, the parameters in Eq. (17) are wavelength dependent. One can choose different wavelengths or multiple wavelengths to measure the blood speed. Since measurements using different wavelengths would make equation (17) overdetermined, one can use least square method to determine the most likely value of speed to minimize measurement errors. In Eq. (16), g (r,a,θ,∞) is the asymptotic value of g₁ when τ approaches infinity, and its value is a function of detector position and blood vessel diameter. In a separate study that solves equation (9) numerically, we have validated the approximations that led to the analytic solution for g₁ in equation (16) and, most importantly, the linear relation between

$$\frac{1}{{T}_{F}}$$

and the blood flow velocity. We have also shown that the decorrelation time for 2D and 3D analyses is nearly the same.

From the approximated relation in equation (17), we find that the characteristic decorrelation rate is proportional to the flow speed of blood. The proportionality constant has a square root dependence on the ratio between the scattering coefficient and the absorption coefficient. This is intuitive since scattering events cause dephasing, and light absorption would terminate the scattering process.

Finally, while the above analysis and experimental setup work for reflected light where the light source(s) and the detector(s) are on the same side with respect to the blood vessel, the result in Eq. 17 can also be applied to the transmitted light. This generalization becomes obvious from FIG. 7 with the angle θ greater than 90 degrees. Here θ is the angle between the axis of light incidence and the axis of light detection.

The characteristic decorrelation rate (1/T_{F}) can be obtained, e.g., by calculating the first inflection point of the curves in FIGS. 2A, 3A, and 4A. The behavior of the curves at longer time is more complicated and can be explained by other slower decorrelation processes than convection such as shear induced diffusion, thus yielding two superimposed Fermi-like functions as shown in FIGS. 2A, 3A, and 4A. To use the model described above, we need to focus on the short time behavior driven by convection.

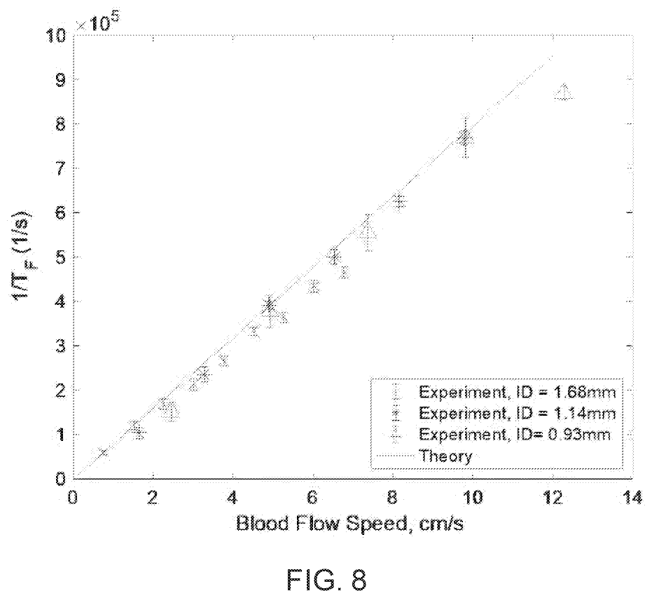

FIG. 8 shows the calculated and measured characteristic decorrelation rates under different flow speeds and tube diameters. An excellent agreement between theory and experiment was achieved, confirming that in the regime where convective flow is the dominating factor for decorrelation, the characteristic decorrelation rate is proportional to the blood speed and independent of the vessel diameter.

FIG. 8. shows a comparison between the theoretical calculations (from Eq. 17) and experimentally measured decorrelation rates under different blood flow speeds and tube diameters. The scattered data set with the error bar represents the 95% confidence interval over 25 measurements. Each measurement took 1 second. The following parameters are used in the calculations: n=1.36, μ′ₛⁱⁿ=1600 m⁻¹ and μₐⁱⁿ=1000 m⁻¹ at 784 nm for deoxygenated blood.

Systems and methods according to the disclosed technology allow performing direct measurements of the blood flow speed in main arteries based on the diffused light model described above. A device according to an example embodiment may use a single fiber bundle, a diode laser, and a photoreceiver. Experimental measurements were performed using a phantom which includes intralipid hydrogel to model the biological tissue and a glass tube embedded into the intralipid hydrogel with human blood flowing through the tube to model a blood vessel. The correlation function of the measured photocurrent was used, according to an example embodiment, to find the electric field correlation function via the Siegert Relation. Notably, the measured electric field correlation function g₁(τ) shows a relation similar to the Fermi-Dirac function, allowing us to define the ln(T_{F}), equivalent to the “Fermi energy” occurring at the first inflection point of g₁(τ). Surprisingly, the value 1/T_{F}, determined according to the present disclosure, which we call characteristic decorrelation rate, is found to be linearly proportional to the blood speed and is independent of the diameter of a blood vessel over the diameters and blood speed ranges for major arteries. This striking property can be explained by an approximate analytic solution for the diffused light equation in the regime where the convective flow dominates the decorrelation. This discovery is highly significant because, for the first time, we can use a device to directly measure the blood speed in major blood vessels (e.g., arteries) without any prior knowledge or assumption about the geometry or mechanical properties of the blood vessels. Non-invasive methods of measuring arterial blood speed according to the present disclosure produce important information about health conditions and provide a new modality for measurements of blood supplies to vital organs.

An implementation of an example method for blood flow speed measurements in a blood vessel according to the disclosed technology includes illuminating an area of a body related to the blood vessel using a light source such that light generated by the light source interacts with blood in the blood vessel. In some example embodiments, interaction of the light generated by the light source with the blood in the blood vessel includes scattering of the light by constituents of the blood such as red blood cells, for example. The example method further includes receiving light that interacted with the blood in the blood vessel by a light detector (e.g., a photodetector), and generating, by the light detector, one or more signals (e.g., a photocurrent signal) using the received light. The one or more signals may include ones that are indicative of temporal variations of one or more properties (e.g., intensity or electric field) of the received light. The example method also includes obtaining a numeric value indicative of the blood flow speed in the blood vessel using an autocorrelation function of at least one signal in the one or more generated signals. For example, an autocorrelation function of the generated photocurrent signal can be used for that purpose. Some implementations of the example method include determining an autocorrelation function of electric field associated with the light received by the light detector by, for example, using the autocorrelation function of the generated photocurrent signal according to the Siegert Relation, and further include obtaining the numeric value indicative of the blood flow speed in the blood vessel using the autocorrelation function of the electric field associated with the light received by the light detector.

The disclosed embodiments illustrate that a characteristic decorrelation rate (i.e. an inverse of a characteristic decoherence or decorrelation time) determined using an autocorrelation function of electric field (e.g., an autocorrelation function of the electric field associated with light received by a light detector, wherein the light received by the light detector has interacted with blood in a blood vessel) disclosed herein can be configured to provide a generally linear relationship between the characteristic decorrelation rate and the blood flow speed in a blood vessel, wherein the relationship is independent from geometric (e.g., diameter) and/or mechanical properties of the blood vessel. Accordingly, systems and methods according to some example embodiments do not include using any prior knowledge, information or assumptions about geometric or mechanical properties of the blood vessel. In some example embodiments, the characteristic decorrelation time corresponds to the first inflection point of the autocorrelation function of the electric field. According to some example embodiments, the inflection point is determined based on the autocorrelation function of the electric field that is in a logarithmic scale along a time axis or variable. The present patent document describes, among other aspects of the disclosed technology, a non-invasive method of measuring blood flow speed in blood vessels without any prior knowledge, information or assumption about geometric or mechanical properties of the blood vessels, thereby greatly simplifying determination of the blood flow speed.

Furthermore, in some example embodiments, the light detector can receive a portion of the light that interacted with the blood in the blood vessel and was reflected and/or scattered by the blood towards the light detector. In such a configuration, the light detector is generally positioned on a same side as the light source relative to the blood vessel. In other example embodiments, the light detector can receive a portion of the light that interacted with the blood in the blood vessel and passed through the blood vessel (e.g., in a direction substantially different from that of the blood flow in the blood vessel). In the latter case, the light detector and the light source may be positioned on the opposite sides relative to the blood vessel. In yet another example implementation of the disclosed technology, the light detector may receive both the light reflected and/or scattered by the blood in the blood vessel and the light transmitted through the blood vessel.

Measurements performed using systems and methods according to some example embodiments can be performed over time intervals on the order of microseconds. We should note that duration of a time interval that is sufficient to perform a measurement according to the technology disclosed herein can be a function of the blood flow speed and can be shorter (or much shorter) or longer (or much longer) compared to the time intervals on the order of microseconds just mentioned. Given that characteristic changes in the blood flow speed in blood vessels (e.g., main arteries and/or veins) caused by heartbeats, for example, or by other physiologic processes in the body, typically occur on a time scale from 0.1 second to many seconds or even minutes or hours, the methods and devices according to the present disclosure are well-suited for monitoring those changes in the blood flow speed. Also, due to a short time scale required to perform a measurement, devices according to the disclosed technology can perform multiple measurements per second and use those multiple measurements to substantially improve signal-to-noise ratio of the generated signals. The above-mentioned features of the disclosed technology make it particularly suited for continuous monitoring of the blood flow speed in various blood vessels of a body.

In some example embodiments, the light source used by devices, systems, and methods according to the disclosed technology can be a light source capable of emitting light predominantly on a single wavelength. For example, such light source can be a laser light source configured to emit light at 784 nm wavelength. Implementations of the technology disclosed herein can also use a light source capable of emitting light on several different wavelengths. As another option, several light sources each working on a certain wavelength can be used. Furthermore, alternatively or in addition to using light sources that emit light predominantly on a single wavelength, light sources capable of emitting light in a portion of the light spectrum can be used by devices, systems and methods according to the technology disclosed herein. For example, a light source that can be used by devices, systems and methods according to some example embodiments can be configured to emit light in a range of wavelengths such that each wavelength in that range is above 200 nm. As another example, a light source that can be used by an example embodiment can be configured to emit light in a range of wavelength such that each wavelength in the range is between 200 nm and 2000 nm. Similarly, systems, devices, and methods according to the technology disclosed in this patent document can use multiple light detectors. For example, an example embodiment of a system according to the disclosed technology can include a light detector positioned to receive light reflected or scattered by the blood in a blood vessel as well as another detector positioned to receive light that passed through the blood vessel. Light detectors used in various embodiments can be positioned at different distances from the blood vessel as well as at different angles around the blood vessel and/or at different angles relative to a normal to skin tissue in front of the blood vessel.

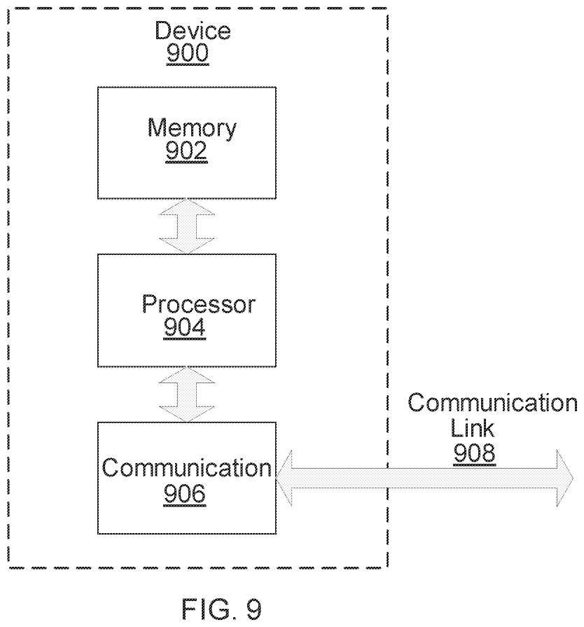

FIG. 9 illustrates a block diagram of a device 900 which can be used to implement, at least in-part, some of the various disclosed embodiments. The device in FIG. 9 can, for example, be implemented as part of the system illustrated in FIG. 1. The device 900 comprises at least one processor and/or controller 904, at least one memory unit 902 that is in communication with the processor 904, and at least one communication unit 906 that enables the exchange of data and information, directly or indirectly, through the communication link 908 with other entities, devices, databases and networks. The communication unit 906 may provide wired and/or wireless communication capabilities in accordance with one or more communication protocols, and therefore it may comprise a transmitter, a receiver or a transceiver, antennas, circuitry, and ports, as well as the encoding/decoding capabilities that may be necessary for transmission and/or reception of data and other information. The example device 900 of FIG. 9 may be integrated as part of any device or system according to the disclosed technology (e.g., as part of the system 100 shown in FIG. 1), to carry out any of the disclosed methods, including receiving information and/or electrical signals corresponding to reflected and/or transmitted light after interaction with the blood, and processing those signals and information to determined blood flow speed. The example device 900 may also be used to control the operation of the light source(s) and/or the detectors of devices and systems according to some example embodiments (e.g., those of the system 100 shown in FIG. 1).

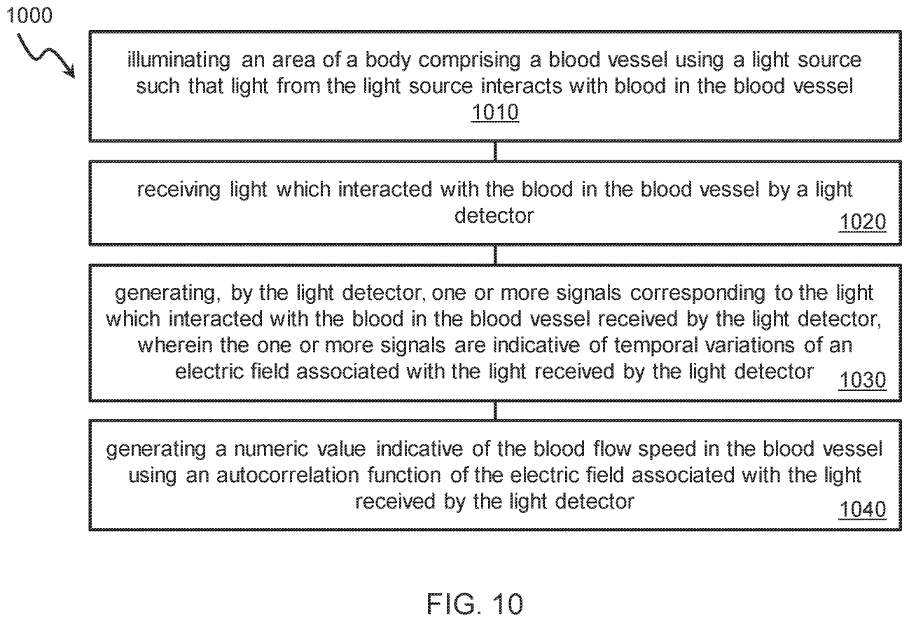

FIG. 10 shows a flow diagram of an example embodiment of a method 1000 of non-invasive blood flow speed measurement in a blood vessel according to the disclosed technology. The method 1000 includes a process 1010 of illuminating an area of a body comprising the blood vessel using a light source such that light from the light source interacts with blood in the blood vessel. The method 1000 further includes a process 1020 of receiving light which interacted with the blood in the blood vessel by a light detector. The method 1000 also includes a process 1030 of generating, by the light detector, one or more signals corresponding to the light which interacted with the blood in the blood vessel received by the light detector, wherein the one or more signals are indicative of temporal variations of an electric field associated with the light received by the light detector. Furthermore, the method 1000 includes a process 1040 of generating a numeric value indicative of the blood flow speed in the blood vessel using an autocorrelation function of the electric field associated with the light received by the light detector.

An aspect of the disclosed embodiments relates to a method of non-invasive blood flow speed measurement in a blood vessel, comprising: illuminating an area of a body comprising the blood vessel using a light source such that light from the light source interacts with blood in the blood vessel; receiving light which interacted with the blood in the blood vessel by a light detector; generating, by the light detector, one or more signals corresponding to the light which interacted with the blood in the blood vessel received by the light detector, wherein the one or more signals are indicative of temporal variations of an electric field associated with the light received by the light detector; generating a numeric value indicative of the blood flow speed in the blood vessel using an autocorrelation function of the electric field associated with the light received by the light detector.

In some example embodiments, said generating the numeric value is performed without using any dimension of the blood vessel or any mechanical property of the blood vessel. According to some example embodiments, interaction of the light from the light source with the blood includes scattering of the light by constituents of the blood. In an example embodiment, the constituents of the blood include red blood cells. According to an example embodiment, the blood vessel is an artery or a vein. In some example embodiments, the one or more signals include a photocurrent signal. An example embodiment comprises determining the autocorrelation function of the electric field using an autocorrelation function of the photocurrent signal. According to some example embodiments, the method further comprises determining an inflection point of the autocorrelation function of the electric field. In an example embodiment, the autocorrelation function of the electric field is on a logarithmic scale with respect to a time variable. In some example embodiments, the inflection point of the autocorrelation function of the electric field is the first inflection point of the autocorrelation function of the electric field. According to some example embodiments, the method further comprises determining a time delay corresponding to the inflection point, wherein said generating the numeric value indicative of the blood flow speed in the blood vessel comprises using the time delay. In an example embodiment, a duration of the non-invasive blood flow speed measurement in the blood vessel is on the order of microseconds. Certain example embodiments of the method comprise generating one or more additional numeric values indicative of the blood flow speed in the blood vessel and further comprise determining the blood flow speed using the numeric value and the one or more additional numeric values. In some example embodiments, the light source is configured to emit light predominantly on a single wavelength. An example embodiment of the method comprises illuminating the blood in the blood vessel using two or more different wavelengths of light, wherein said generating the numeric value indicative of the blood flow speed is performed based on measurements conducted using the two or more different wavelengths of light that illuminates the blood in the blood vessel. In some example embodiments, said receiving the light which interacted with the blood in the blood vessel by the light detector comprises receiving the light which interacted with the blood in the blood vessel which was reflected or scattered back by the blood. According to some example embodiments, said receiving the light which interacted with the blood in the blood vessel by the light detector comprises receiving the light which interacted with the blood in the blood vessel which has transmitted through the blood. In an example embodiment, interaction of the light from the light source with the blood in the blood vessel includes scattering of the light by constituents of the blood in the blood vessel. In some example embodiments, the constituents of the blood in the blood vessel include red blood cells in the blood vessel. According to some example embodiments, said generating the numeric value indicative of the blood flow speed in the blood vessel comprises determining an inflection point of the autocorrelation function of the electric field. In an example embodiment, the light source is a laser or a light-emitting diode (LED) light source. In some example embodiments, said illuminating the area of the body comprises illuminating the area of the body using two or more different wavelengths of light, and wherein said generating the numeric value indicative of the blood flow speed in the blood vessel is performed based on measurements conducted based on the two or more different wavelengths of light.

Another aspect of the disclosed embodiments relates to a system for non-invasive blood speed measurements, comprising: one or more light sources configured to produce light to illuminate an area of a body comprising a blood vessel; one or more light detectors positioned to receive light which interacted with blood in the blood vessel and generate one or more signals corresponding to received light which interacted with the blood in the blood vessel and indicative of temporal variations of an electric field associated with the received light; a processor; and a memory comprising processor executable code, wherein the processor executable code, upon execution by the processor, causes the processor to perform operations comprising: determining an estimate of a blood flow speed in the blood vessel using an autocorrelation function of the electric field associated with the received light.

In some example embodiments, the operations comprise producing the autocorrelation function of the electric field using an autocorrelation function of a photocurrent signal. According to some example embodiments, the operations comprise determining a time delay corresponding to an inflection point of the autocorrelation function of the electric field, wherein said determining the estimate of the blood flow speed in the blood vessel comprises using the time delay. In an example embodiment, the inflection point of the autocorrelation function of the electric field is the first inflection point of the autocorrelation function of the electric field. In some example embodiments, said determining the estimate of the blood flow speed is performed without using any dimension of the blood vessel or any mechanical property of the blood vessel. According to some example embodiments, the one or more light sources are configured to illuminate the blood in the blood vessel at two or more different wavelengths of light and wherein said determining the estimate of the blood flow speed in the blood vessel is performed based on measurements conducted using the two or more different wavelengths of light that illuminates the blood in the blood vessel. In an example embodiment, the one or more light detectors are positioned to receive light after reflection or scattering of the light by the blood. In another example embodiment, the one or more light detectors are positioned to receive light after transmission of the light through the blood.

Yet another aspect of the disclosed embodiments relates to a system for non-invasive blood speed measurements, comprising: one or more light sources configured to produce light to illuminate an area of a body comprising a blood vessel; one or more light detectors positioned to receive light subsequent to interaction with blood in the blood vessel and to generate one or more signals corresponding to received light after interaction with the blood in the blood vessel, the one or more signals indicative of temporal variations of an electric field associated with the received light; a processor coupled to the one or more light detectors; and a memory comprising processor executable code, wherein the processor executable code, upon execution by the processor, causes the processor to: receive information corresponding to the one or more signals generated by the one or more light detectors and determine an estimate of a blood flow speed in the blood vessel using an autocorrelation function of the electric field associated with the received light without using any dimension of the blood vessel or any mechanical property of the blood vessel.

In some example embodiments, the one or more signals include a photocurrent signal, the information corresponding to the one or more signals includes information related to the photocurrent signal, and wherein the processor executable code, upon execution by the processor, causes the processor to compute the autocorrelation function of the electric field using an autocorrelation function of the photocurrent signal determined, by the processor, using the information related to the photocurrent signal. According to some example embodiments, the processor executable code, upon execution by the processor, causes the processor to compute a time delay corresponding to an inflection point of the autocorrelation function of the electric field, and wherein said determine the estimate of the blood flow speed in the blood vessel is performed using the time delay. In an example embodiment, the one or more light sources are configured to illuminate the area of the body at two or more different wavelengths of light and wherein said determine the estimate of the blood flow speed in the blood vessel is performed based on measurements conducted based on the two or more different wavelengths of light.

An aspect of the disclosed embodiments relates to a method for non-invasive blood flow speed measurement in a blood vessel, comprising: illuminating an area of a body comprising the blood vessel using a light source such that light from the light source interacts with blood in the blood vessel; receiving light after interaction with the blood in the blood vessel by a light detector; obtaining one or more signals corresponding to the received light from the light detector, wherein the one or more signals are indicative of temporal variations of an electric field associated with the received light; obtaining a numeric value indicative of the blood flow speed in the blood vessel using an autocorrelation function of an electric field associated with the light received by the light detector.

In some example embodiments, said obtaining the numeric value does not include using any prior knowledge or assumptions about geometric or mechanical properties of the blood vessel. According to an example embodiment, interaction of the light with the blood includes scattering of the light by constituents of the blood. In an example embodiment, said obtaining the numeric value includes obtaining the autocorrelation function of the electric field associated with a photocurrent signal. In some example embodiments, the blood vessel is a vein or an artery. According to some example embodiments, the method further comprises determining a time delay corresponding to an inflection point of the autocorrelation function of the electric field and using the time delay to obtain the numeric value indicative of the blood flow speed in the blood vessel. In an example embodiment, the inflection point is determined based on the autocorrelation function of the electric field that is in a logarithmic scale. In some example embodiments, the inflection point of the autocorrelation function of the electric field is the first inflection point of the autocorrelation function of the electric field. According to some example embodiments, a duration of blood flow speed measurement in the blood vessel is in the order of microseconds. In an example embodiment, the method further comprises obtaining one or more additional numeric values indicative of the blood flow speed, and determining the blood flow speed with a higher accuracy using a combination of the one or more additional numeric values. In some example embodiments, the method also comprises illuminating the blood in the blood vessel using two or more different wavelengths of light and obtaining the numeric value indicative of the blood flow speed based on measurements conducted using the two or more different wavelengths of light that illuminates the blood in the blood vessel. According to some example embodiments, said receiving the light by the light detector includes receiving the light (a) that has transmitted through the blood, or (b) that was reflected or scattered back by the blood.

Another aspect of the disclosed embodiments relates to a system for non-invasive blood speed measurements, comprising: one or more light sources configured to produce light to illuminate an area of a body comprising a blood vessel; one or more light detectors positioned to receive light that interacted with the blood in the blood vessel and generate one or more signals corresponding to the received light and indicative of temporal variations of an electric field associated with the received light; a processor; and a memory comprising processor executable code, wherein the processor executable code, upon execution by the processor, causes the processor to: determine an estimate of the blood flow speed in the blood vessel using an autocorrelation function of an electric field associated with the light received by the light detector.

In some example embodiments, the blood vessel is a vein or an artery. According to some example embodiments, the processor executable code, upon execution by the processor, also causes the processor to: obtain the autocorrelation function of the electric field using an autocorrelation function of a photocurrent signal. In an example embodiment, the processor executable code, upon execution by the processor, also causes the processor to: determine a time delay corresponding to an inflection point of the autocorrelation function of the electric field; and use the time delay to compute the estimate of the blood flow speed in the blood vessel. In a certain example embodiment, the inflection point of the autocorrelation function of the electric field is the first inflection point of the autocorrelation function of the electric field. In some example embodiments, the processor executable code, upon execution by the processor, also causes the processor to: determine the estimate of the blood flow speed without using any dimension of the blood vessel or any mechanical property of the blood vessel. According to some example embodiments, interaction of the light with the blood includes scattering of the light by one or more constituents of the blood. In an example embodiment, a duration of blood flow speed measurement in the blood vessel is in the order of microseconds. In some example embodiments, the processor executable code, upon execution by the processor, also causes the processor to obtain one or more additional numeric values indicative of the blood flow speed, and determine the blood flow speed with a higher accuracy using a combination of the one or more additional numeric values. According to an example embodiment, the one or more light sources are configured to illuminate the blood in the blood vessel at two or more different wavelengths of light and wherein the processor executable code, upon execution by the processor, also causes the processor to obtain the numeric value indicative of the blood flow speed based on measurements conducted using the two or more different wavelengths of light that illuminates the blood in the blood vessel. In some example embodiments, the one or more light detectors are positioned to receive the light (a) after transmission through the blood, or (b) after reflection or scattering by the blood.

Some of the disclosed devices or modules can be implemented as hardware, software, or combinations thereof. For example, a hardware implementation of electronic devices can include discrete analog and/or digital components that are, for example, integrated as part of a printed circuit board. Alternatively, or additionally, the disclosed components or modules can be implemented as an Application Specific Integrated Circuit (ASIC) and/or as a Field Programmable Gate Array (FPGA) device. Some implementations may additionally or alternatively include a digital signal processor (DSP) that is a specialized microprocessor with an architecture optimized for the operational needs of digital signal processing associated with the disclosed functionalities of this application. Similarly, the various components or sub-components within each module may be implemented in software, hardware or firmware. The connectivity between the modules and/or components within the modules may be provided using any one of the connectivity methods and media that are known in the art, including, but not limited to, communications over the Internet, wired, or wireless networks using the appropriate protocols.

Various information and data processing operations described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

What is claimed is:

1. A method of non-invasive blood flow speed measurement in a blood vessel, comprising:
illuminating an area of a body comprising the blood vessel using a light source such that light from the light source interacts with blood in the blood vessel;
receiving light which interacted with the blood in the blood vessel by a light detector;
generating, by the light detector, one or more signals corresponding to the light which interacted with the blood in the blood vessel received by the light detector, wherein the one or more signals are indicative of temporal variations of an electric field associated with the light received by the light detector;
generating a numeric value indicative of the blood flow speed in the blood vessel using an autocorrelation function of the electric field associated with the light received by the light detector,
wherein said generating the numeric value is performed without using any dimension of the blood vessel or any mechanical property of the blood vessel.

2. The method of claim 1, wherein interaction of the light from the light source with the blood in the blood vessel includes scattering of the light by constituents of the blood in the blood vessel.

3. The method of claim 2, wherein the constituents of the blood in the blood vessel include red blood cells in the blood vessel.

4. The method of claim 1, wherein the blood vessel is an artery or a vein.

5. The method of claim 1, wherein the one or more signals include a photocurrent signal, and wherein the method further comprises determining the autocorrelation function of the electric field using an autocorrelation function of the photocurrent signal.

6. The method of claim 1, wherein said generating the numeric value indicative of the blood flow speed in the blood vessel comprises determining an inflection point of the autocorrelation function of the electric field.

7. The method of claim 6, wherein the autocorrelation function of the electric field is on a logarithmic scale with respect to a time variable.

8. The method of claim 6, wherein the inflection point of the autocorrelation function of the electric field is the first inflection point of the autocorrelation function of the electric field.

9. The method of claim 6, comprising:
determining a time delay corresponding to the inflection point,
wherein said generating the numeric value indicative of the blood flow speed in the blood vessel comprises using the time delay.

10. The method of claim 1, wherein a duration of the non-invasive blood flow speed measurement in the blood vessel is on the order of microseconds.

11. The method of claim 1, comprising:
generating one or more additional numeric values indicative of the blood flow speed in the blood vessel; and
determining the blood flow speed using the numeric value and the one or more additional numeric values.

12. The method of claim 1, wherein the light source is a laser or a light-emitting diode (LED) light source.

13. The method of claim 1, wherein said illuminating the area of the body comprises illuminating the area of the body using two or more different wavelengths of light, and wherein said generating the numeric value indicative of the blood flow speed in the blood vessel is performed based on measurements conducted based on the two or more different wavelengths of light.

14. The method of claim 1, wherein said receiving the light which interacted with the blood in the blood vessel by the light detector comprises receiving the light which interacted with the blood in the blood vessel which was reflected or scattered back by the blood.

15. The method of claim 1, wherein said receiving the light which interacted with the blood in the blood vessel by the light detector comprises receiving the light which interacted with the blood in the blood vessel which has transmitted through the blood.

16. A system for non-invasive blood speed measurements, comprising:
one or more light sources configured to produce light to illuminate an area of a body comprising a blood vessel;
one or more light detectors positioned to receive light subsequent to interaction with blood in the blood vessel and to generate one or more signals corresponding to received light after interaction with the blood in the blood vessel, the one or more signals indicative of temporal variations of an electric field associated with the received light;
a processor coupled to the one or more light detectors; and
a memory comprising processor executable code, wherein the processor executable code, upon execution by the processor, causes the processor to:
receive information corresponding to the one or more signals generated by the one or more light detectors and determine an estimate of a blood flow speed in the blood vessel using an autocorrelation function of the electric field associated with the received light without using any dimension of the blood vessel or any mechanical property of the blood vessel.

17. The system of claim 16, wherein the one or more signals include a photocurrent signal, the information corresponding to the one or more signals includes information related to the photocurrent signal, and wherein the processor executable code, upon execution by the processor, causes the processor to compute the autocorrelation function of the electric field using an autocorrelation function of the photocurrent signal determined, by the processor, using the information related to the photocurrent signal.

18. The system of claim 16, wherein the processor executable code, upon execution by the processor, causes the processor to compute a time delay corresponding to an inflection point of the autocorrelation function of the electric field, and wherein said determine the estimate of the blood flow speed in the blood vessel is performed using the time delay.

19. The system of claim 18, wherein the inflection point of the autocorrelation function of the electric field is the first inflection point of the autocorrelation function of the electric field.

20. The system of claim 16, wherein the one or more light sources are configured to illuminate the area of the body at two or more different wavelengths of light and wherein said determine the estimate of the blood flow speed in the blood vessel is performed based on measurements conducted based on the two or more different wavelengths of light.

21. The system of claim 16, wherein the one or more light detectors are positioned to receive light after reflection or scattering of the light by the blood.

22. The system of claim 16, wherein the one or more light detectors are positioned to receive light after transmission of the light through the blood.
